(19) United States
(12) Patent Application Publication
(10) Pub. No.: US 2017/0087280 A1
(43) Pub. Date: Mar. 30, 2017

(54) SPATIOTEMPORALLY MULTICONTROLLABLE INTELIGENT DRUG-ELUTING STENT

(71) Applicant: KOREA INSTITUTE OF SCIENCE AND TECHNOLOGY, Seoul (KR)

(72) Inventors: Dongkeun HAN, Seoul (KR); Yoonki JOUNG, Seoul (KR); Jonghee KANG, Seoul (KR); Eugene LIH, Seoul (KR); Seongmin KIM, Seoul (KR)

(21) Appl. No.: 15/041,239

(22) Filed: Feb. 11, 2016

(30) Foreign Application Priority Data
Sep. 24, 2015 (KR) 10-2015-0135174

(51) Int. Cl. A61L 31/16; A61L 31/10; A61K 31/137; A61K 31/436; A61K 33/08; A61K 31/616; A61K 31/4375; A61K 31/385; C07K 16/28; A61K 31/727; A61K 35/644; A61K 31/337; A61K 31/192; A61K 31/4365; A61K 31/196; A61K 31/519; A61K 33/06; A61K 31/565; A61K 38/07; A61K 33/00; A61K 31/343; A61L 31/14; A61L 31/02

(52) U.S. Cl. CPC A61L 31/16; A61L 31/022; A61L 31/10; A61K 31/137; A61K 31/436; A61K 33/08; A61K 31/616; A61K 31/4375; A61K 31/385; C07K 16/2848; A61K 31/727; A61K 35/644; A61K 31/337; A61K 31/192; A61K 31/4365; A61K 31/196; A61K 31/519; A61K 33/06; A61K 31/565; A61K 38/07; A61K 33/00; A61K 31/343; A61L 31/148; C07K 2317/24; A61L 2300/41; A61L 2300/416; A61L 2420/08; A61L 2300/42

(57) ABSTRACT

The present invention relates to a spatiotemporally multicontrollable intelligent drug-eluting adjustable stent, and a manufacturing method thereof, and includes a stent, a nano-coupling layer formed on the inner and outer surfaces of the stent and including a bioactive material into which a catechol group-containing adhesive derivative is introduced, and a second coating layer formed on the upper portion of the first coating layer and on the nano-coupling layer at the inner portion of the stent, and including an antithrombotic agent and an anti-inflammatory agent.

### CROSS-REFERENCE TO RELATED APPLICATION

Pursuant to 35 U.S.C. §119(a), this application claims the benefit of earlier filing date and right of priority to Korean Application No. 10-2015-0135174, filed Sep. 24, 2015, the contents of which is incorporated by reference herein in its entirety.

### TECHNICAL FIELD

The present invention relates to a spatiotemporally multicontrollable intelligent drug-eluting adjustable stent, and a manufacturing method thereof. More particularly, the present invention relates to an intelligent drug-eluting adjustable stent, which significantly lowers side effects in the initial stage, the middle stage, and the late stage after a stent implantation by coating different drugs and bioactive materials on the inner and outer surfaces of the stent, such that the biodegradation period and the release time may be adjusted.

### BACKGROUND ART

A stent refers to a mesh tube implanted when the inner diameter of the artery or the blood vascular system becomes so narrow due to deposition of thrombus or lipids in the coronary arteries and the peripheral blood vessels that the flow of the bloodstream is not smooth, or when tumors occur in the non-vascular system such as the gastrointestinal tract, the esophagus, and the respiratory tract, or against stenosis occurring after a surgery. The major problems of a stent implantation are the occurrences of in-stent restenosis in which the coronary arteries are again stenosed after the stent surgery and stent thrombosis. Further, inflammation due to the damage to the endothelium of the blood vessels during the process of the treatment is also commonly occurred problem.

Currently, in order to prevent restenosis, a stent supplying a cardiovascular or lumen wall support or reinforcement has been widely used, but it is known that approximately 20 to 30% of blood vessels or lumen walls of the patients with the stent are reoccluded within several weeks and several months.

In addition, in the case of a stent made of a metal material, an excessive neointima formation resulting from the damage of the vascular endothelial wall during the implant process occurs, and is also responsible for the restenosis. As a recent method used to address the problem of neointimal hyperplasia, a drug-eluting stent manufactured by coating a stent with an immunosuppressant or a chemotherapeutic agent has been widely used. Specifically, there has been developed a drug-eluting antithrombogenic multi-layer coated stent composed of a lower layer, which is a primary coating layer in which a biologically active material is distributed, and an upper layer, which is a secondary coating layer in which a hydrophobic heparin polymer having antithrombogenic properties is distributed (Korean Patent Application Laid-Open No. 10-2004-0028486).

Meanwhile, heparin extracted from porcine intestines is a material which is well-known to have an anti-clotting capacity, and may inhibit both restenosis and thrombosis, and thus is used as a drug to be coated on a stent. Specifically, U.S. Pat. No. 5,837,313 (Ding et al.) describes a technology in which the surface of a stent is treated with a thin polymer loaded with heparin, as a method of preparing heparin as a coating composition.

Furthermore, in order for preventing neointimal hyperplasia, a multilayer-coated stent for controlled drug release has been developed, and specifically, there has been known multilayer-coated stent for controlled drug release comprising: a first base layer formed on a stent support and made of a poly(ethylene-co-vinyl acetate) or styrene-based rubber polymer; a second coating layer formed on the first base layer and made of a biocompatible polymer and a drug component; and a third coating layer formed on the second coating layer and made of a biocompatible polymer and drug component different from the drug component of the second coating layer (Korean Patent No. 10-0511618).

Further, as a measure for reducing late stent thrombosis, there is also known a drug-eluting stent having a metal body whose outer surfaces are treated to enhance the adhesion of a biodegradable polymer coating by applying a coating of non-degradable polymer parylene to the surfaces and having on the treated outer surfaces, a drug-polymer coating formulated to contain a anti-restenosis drug (Korean Patent Application Laid-Open No. 10-2011-0048021).

### DISCLOSURE OF THE INVENTION

However, since all the conventional technologies fail to reach a level of precisely controlling the elution of a necessary drug depending on the period after a stent implantation, there is still a need for the technology of precisely controlling the elution.

Therefore, an object of the present invention is to provide a drug-elution controlling stent which may significantly reduce side effects occurring at the initial, middle, and late stages after a stent implantation by precisely adjusting the biodegradation and release time of a coating material on the surface of the stent in order to efficiently overcome occurrence of restenosis, thrombosis, and inflammation, and the like, which are the side effects resulting from the stent implantation. In addition, another object of the present invention is to provide a smooth coating surface in order to solve problems such as the instability of a coating layer when the surface of a metal stent is coated with a polymer and a drug, and the occurrence of cracks on the surface of the stent when ballooning is performed during the process of the stent insertion treatment, and to provide a stable stent coating method capable of preventing cracks from being formed on the surface of the coating.

To achieve these and other advantages and in accordance with the purpose of the present invention, as embodied and broadly described herein, there is provided a spatiotemporally multicontrollable intelligent drug-eluting adjustable stent according to the present invention. Specifically, the configuration of the stent provided by the present invention is as follows.

(1) A drug-eluting stent including:

a stent;

a nano-coupling layer formed on the inner and outer surfaces of the stent and including a bioactive material into which a catechol group-containing adhesive derivative is introduced;

a first coating layer formed on the nano-coupling layer on the outer surface of the stent and including a restenosis preventing agent and an anti-inflammatory agent, which are mixed with a biocompatible and biodegradable polymer; and

a second coating layer formed on the first coating layer on the outer surface of the stent and on the nano-coupling layer on the inner surface of the stent, and including an antithrombotic agent and an anti-inflammatory agent, which are mixed with a biocompatible and biodegradable polymer.

(2) The drug-eluting stent of (1), in which the stent is made of a metal material selected from the group consisting of stainless steel, cobalt-chromium, platinum-chromium, tantalum, titanium, nitinol, platinum-iridium, iron, gold, platinum, silver, magnesium, and alloys thereof, or a polymer material selected from the group consisting of poly(glycolic acid) (PGA), poly(L-lactic acid) (PLLA), poly(D-lactic acid) (PDLA), poly(D,L-lactic acid) (PDLLA), poly(c-caprolactone) (PCL), poly(lactic acid-co-glycolic acid) (PLGA), poly(L-lactic acid-co-c-caprolactone) (PLCL), tyrosine polycarbonate, salicylic acid-containing polymers, polyethylene glycol, polyamino acid, polyanhydride, polyorthoester, polydioxanone, polyphosphazene, cellulose acetate butyrate, cellulose triacetate, and copolymers thereof.

(3) The drug-eluting stent of (1), in which the bioactive material of the nano-coupling layer is selected from the group consisting of hyaluronic acid (HA), acetylated hyaluronic acid (AcHA), chondroitin sulfate (CS), heparin (HEP), heparan sulfate (HS), dextran (DEX), dextran sulfate (DEXS), dermatan sulfate (DS), keratan sulfate (KS), vita-hyaluronic acid (vita-HA), vita-acetylated hyaluronic acid (vita-AcHA), vita-chondroitin sulfate (vita-CS), vita-heparin (vita-HEP), vita-heparan sulfate (vita-HS), vita-dextran (vita-DEX), vita-dextran sulfate(vita-DEXS), vita-dermatan sulfate (vita-DS), and vita-keratan sulfate (vita-KS).

The vita-hyaluronic acid, vita-acetylated hyaluronic acid, vita-chondroitin sulfate, vita-heparin, vita-heparan sulfate, vita-dextran, vita-dextran sulfate, vita-dermatan sulfate, and vita-keratan sulfate mean that any one of vitamin A, a vitamin B complex, vitamin C, vitamin D, vitamin E, vitamin F, vitamin K, vitamin U, vitamin L, and vitamin P each combines with hyaluronic acid, acetylated hyaluronic acid, chondroitin sulfate, heparin, heparan sulfate, dextran, dextran sulfate, dermatan sulfate, or keratan sulfate.

(4) The drug-eluting stent of (1), in which the catechol group-containing adhesive derivative of the nano-coupling layer is selected from the group consisting of dopamine (DA), norepinephrine, epinephrine, 3,4-dihydroxybenzylamine, 3,4-dihydroxycinnamic acid, 3,4-dihydroxyphenyl acetic acid, 3,4-dihydroxymandelic acid, 3,4-dihydroxyphenyl lactic acid, 3,4-dihydroxyphenylalanine, 2-(3,4-dihydroxyphenyl)ethanol, 3,4-dihydroxyphenylethyleneglycol, 3,4-dihydroxyphenylacetaldehyde, 3,4-dihydroxyphenylglycol aldehyde, and isoproterenol.

(5) The drug-eluting stent of (1), in which the biocompatible polymer of the first coating layer is a polymer having a molecular weight in a range of 5,000 to 500,000, selected from the group consisting of polyvinyl alcohol, polyethylene glycol, polylactide, polyglycolide, polylactide copolymers, polycaprolactone copolymers, polyethylene oxide, polydioxanone, and polyvinylpyrrolidone.

(6) The drug-eluting stent of (1), in which the restenosis preventing agent of the first coating layer is selected from the group consisting of alpha-lipoic acid, abciximab, sirolimus, sirolimus derivatives, paclitaxel, dexamethasone, tacrolimus, mycophenolic acid, estradiol, taxol, colchicine, lovastatin, trapidil, hirudin, ticlopidine, and nitrogen oxides.

(7) The drug-eluting stent of (1), in which the anti-inflammatory agent of the first coating layer and the second coating layer is one or more selected from the group consisting of magnesium, magnesium oxide, magnesium fluoride, magnesium chloride, magnesium hydroxide, lithium hydroxide, beryllium hydroxide, sodium hydroxide, potassium hydroxide, rubidium hydroxide, strontium hydroxide, barium hydroxide, cesium hydroxide, francium hydroxide, and radium hydroxide.

(8) The drug-eluting stent of (1), in which the anti-inflammatory agent of the second coating layer is one or more selected from the group consisting of vinpocetine, Ac-SDKP, propolis, ibuprofen, diclofenac, and naproxen.

(9) The drug-eluting stent of (1), in which the antithrombotic agent of the second coating layer is one or more selected from the group consisting of aspirin, propolis, heparin, prasugrel, ticagrelor, and clopidogrel.

(10) A method for manufacturing a drug-eluting stent, the method including:

coating a nano-coupling layer including a bioactive material into which a catechol group-containing adhesive derivative is introduced on the inner and outer surfaces of a metal or polymer stent via a dipping coating method;

coating a first coating layer including a restenosis preventing agent and an anti-inflammatory agent, which are mixed with a biocompatible polymer, only on the outer surface of the stent coated with the nano-coupling layer, and

coating a second coating layer including an antithrombotic agent and an anti-inflammatory agent on the first coating layer on the outer surface of the stent and on the nano-coupling layer on the inner surface of the stent.

(11) The method of (1), in which the bioactive material of the nano-coupling layer is selected from the group consisting of hyaluronic acid, acetylated hyaluronic acid, chondroitin sulfate, heparin, heparan sulfate, dextran, dextran sulfate, dermatan sulfate, keratan sulfate, vita-hyaluronic acid, vita-acetylated hyaluronic acid, vita-chondroitin sulfate, vita-heparin, vita-heparan sulfate, vita-dextran, vita-dextran sulfate, vita-dermatan sulfate, and vita-keratan sulfate.

The vita-hyaluronic acid, vita-acetylated hyaluronic acid, vita-chondroitin sulfate, vita-heparin, vita-heparan sulfate, vita-dextran, vita-dextran sulfate, vita-dermatan sulfate, and vita-keratan sulfate mean that any one of vitamin A, a vitamin B complex, vitamin C, vitamin D, vitamin E, vitamin F, vitamin K, vitamin U, vitamin L, and vitamin P each combines with hyaluronic acid, acetylated hyaluronic acid, chondroitin sulfate, heparin, heparan sulfate, dextran, dextran sulfate, dermatan sulfate, or keratan sulfate.

(12) The method of (10), in which the catechol group-containing adhesive derivative of the nano-coupling layer is selected from the group consisting of dopamine, norepinephrine, epinephrine, 3,4-dihydroxybenzylamine, 3,4-dihydroxycinnamic acid, 3,4-dihydroxyphenyl acetic acid, 3,4-dihydroxymandelic acid, 3,4-dihydroxyphenyl lactic acid, 3,4-dihydroxyphenylalanine, 2-(3,4-dihydroxyphenyl)ethanol, 3,4-dihydroxyphenylethyleneglycol, 3,4-dihydroxyphenylacetaldehyde, 3,4-dihydroxyphenylglycol aldehyde, and isoproterenol.

(13) The method of (10), in which the biocompatible polymer of the first coating layer is a polymer having a molecular weight in a range of 5,000 to 500,000, selected from the group consisting of polyvinyl alcohol, polyethylene glycol, polylactide, polyglycolide, polylactide copolymers, polycaprolactone copolymers, polyethylene oxide, polydioxanone, and polyvinylpyrrolidone.

(14) The method of (10), in which the restenosis preventing agent of the first coating layer is selected from the group consisting of alpha-lipoic acid, abciximab, sirolimus, sirolimus derivatives, paclitaxel, dexamethasone, tacrolimus, mycophenolic acid, estradiol, taxol, colchicine, lovastatin, trapidil, hirudin, ticlopidine, and nitrogen oxides.

(15) The method of (10), in which the anti-inflammatory agent of the first coating layer and the second coating layer is one or more selected from the group consisting of magnesium, magnesium oxide, magnesium fluoride, magnesium hydroxide, lithium hydroxide, beryllium hydroxide, sodium hydroxide, potassium hydroxide, rubidium hydroxide, strontium hydroxide, barium hydroxide, cesium hydroxide, francium hydroxide, and radium hydroxide.

(16) The method of (10), in which the anti-inflammatory agent of the second coating layer is one or more selected from the group consisting of vinpocetine, Ac-SDKP, propolis, ibuprofen, diclofenac, and naproxen.

(17) The method of (10), in which the antithrombotic agent of the second coating layer is one or more selected from the group consisting of aspirin, propolis, heparin, prasugrel, ticagrelor, and clopidogrel.

According to the drug-eluting stent of the present invention, drugs having complementary mechanism of action are coated differently on the inner and outer surfaces of the stent to provide a spatiotemporally multicontrollable drug-eluting adjustment, thereby inhibiting formation of initial thrombosis and inflammation resulting from a stent implantation, and it also is possible to prevent occurrence of restenosis and inflammation at the middle stage after the implantation, and to promote reendothelialization and inhibit late stent thrombosis.

The drug-eluting stent according to the present invention is composed of a stent, a nano-coupling layer formed on the inner and outer surfaces of the stent and comprising a bioactive material into which a catechol group-containing adhesive derivative is introduced, a first coating layer formed on the outer surface of the stent and including a restenosis preventing agent and an anti-inflammatory agent, which are mixed with a biocompatible; and a second coating layer formed on the first coating layer on the outer surface of the stent and on the nano-coupling layer on the inner surface of the stent, and including an antithrombotic agent and an anti-inflammatory agent.

According to the configuration, first, the antithrombotic agent and the inflammation inhibitor, which is the drugs of the second coating layer of the uppermost layer of a stent, are released within a week after a stent surgery, thereby inhibiting thrombosis at the initial stage after the stent treatment, and inhibiting inflammation resulting from the damage to the endothelium of the blood vessels during the stent treatment process. Thereafter, the restenosis preventing agent and the anti-inflammatory agent in the first coating layer are released, and the drugs in the first coating layer are completely released and degraded within 3 months after the stent treatment, thereby preventing formation of restenosis at the middle stage after the stent treatment, and inhibiting inflammation resulting from an increase in acidity due to degradation of the biodegradable polymer. After the first drug layer is released, only a nano-coupling layer formed by coating a bioactive material into which a catechol group-containing adhesive derivative is introduced remains on the surface of the stent, and it is possible to prevent occurrence of cracks on the surface of the stent and impart flexibility thereto due to the nano-coupled coating layer. Further, the nano-coupling layer includes a bioactive material, and thus inhibits late stent thrombosis 3 months after the stent implantation, and promotes reendothelialization.

Specifically, the stent according to the present invention may be made of a metal or polymer stent material. As the metal material, it is possible to use a metal selected from group consisting of stainless steel, cobalt-chromium, platinum-chromium, tantalum, titanium, nitinol, platinum-iridium, gold, platinum, silver, magnesium, and alloys thereof. In addition, as the polymer material, it is possible to use a polymer selected from polylactide, polyglycolide, polylactide-polyglycolide copolymers, polydioxanone, polycaprolactone, and polylactide-polycaprolactone copolymers, and the like.

Furthermore, the nano-coupling layer to be formed on the inner and outer surfaces of the stent is formed by dip-coating a bioactive material into which a catechol group-containing adhesive derivative is introduced on both the inner and outer surfaces of the stent. Specifically, the bioactive material is selected from the group consisting of hyaluronic acid, acetylated hyaluronic acid, chondroitin sulfate, heparin, heparan sulfate, dextran, dextran sulfate, dermatan sulfate, keratan sulfate, vita-hyaluronic acid, vita-acetylated hyaluronic acid, vita-chondroitin sulfate, vita-heparin, vita-heparan sulfate, vita-dextran, vita-dextran sulfate, vita-dermatan sulfate, and vita-keratan sulfate. These bioactive materials simultaneously inhibit late stent thrombosis and promote endothelialization.

Further, the catechol group-containing adhesive derivative combining with the bioactive material is selected from the group consisting of dopamine, norepinephrine, epinephrine, 3,4-dihydroxybenzylamine, 3,4-dihydroxycinnamic acid, 3,4-dihydroxyphenyl acetic acid, 3,4-dihydroxymandelic acid, 3,4-dihydroxyphenyl lactic acid, 3,4-dihydroxyphenylalanine, 2-(3,4-dihydroxyphenyl)ethanol, 3,4-dihydroxyphenylethyleneglycol, 3,4-dihydroxyphenylacetaldehyde, 3,4-dihydroxyphenylglycol aldehyde, and isoproterenol.

These bioactive materials into which a catechol group-containing adhesive derivative is introduced, which constitute a nano-coupling layer, are nano-coupled with a metal or polymer stent material by coating or a solution reaction, thereby forming the nano-coupling layer. In addition, coating stability may be improved by forming the nano-coupling layer, cracks may be prevented from being formed by applying flexibility to the coating, and it is possible to inhibit late stent thrombosis and induce reendothelialization.

In particular, the bioactive material of the nano-coupling layer is a material into which a catechol group-containing adhesive derivative is introduced, and since the catechol group-containing adhesive derivative may be substituted in the form of dopaquinone under basic conditions to strongly combine with the stent surface material, adhesion may be improved by a nano-coupling layer, and cracks may be prevented from occurring on the coating of the surface of the stent by imparting flexibility on the coating layer.

Furthermore, a first coating layer including a restenosis preventing agent and an anti-inflammatory agent, which are mixed with a biocompatible polymer; is formed only on the outer surface of the stent, and as the biocompatible polymer, there is used a polymer having a molecular weight in a range of 5,000 to 500,000, preferably 10,000 to 300,000 selected from the group consisting of polyvinyl alcohol, polyethylene glycol, polylactide, polyglycolide, polylactide copolymers, polycaprolactone copolymers, polyethylene oxide, polydioxanone, and polyvinylpyrrolidone.

Also, as the restenosis preventing agent, it is possible to use any one selected from the group consisting of alpha-lipoic acid, abciximab, sirolimus, sirolimus derivatives, paclitaxel, dexamethasone, tacrolimus, mycophenolic acid, estradiol, taxol, colchicine, lovastatin, trapidil, hirudin, ticlopidine, and nitrogen oxides. At this time, the nitrogen oxides are any one selected from monomers and polymer compounds, which contain nitric oxide (NO) such as NONOate. As the inflammation inhibitor, it is possible to use one or more selected from the group consisting of magnesium, magnesium oxide, magnesium fluoride, magnesium chloride, magnesium hydroxide, lithium hydroxide, beryllium hydroxide, sodium hydroxide, potassium hydroxide, potassium hydroxide, rubidium hydroxide, strontium hydroxide, barium hydroxide, cesium hydroxide, francium hydroxide, and radium hydroxide. The anti-inflammatory agent and the restenosis preventing agent are mixed at a ratio of 1 to 50% with a polymer material, and a preferable content of the restenosis preventing agent is 1 to 500 μg.

It is important to note that the first coating layer in the drug-eluting stent of the present invention is coated only on the outer surface of the stent. It is intended to inhibit proliferation of vascular smooth muscle cells and induce apoptosis of vascular smooth muscle cells by coating a restenosis preventing agent which is a cell proliferation inhibitor only on the outer surface thereof in order to prevent restenosis. Since it is necessary to improve the blood compatibility by inducing reendothelialization resulting from the proliferation of endothelial cells and thus prevent vascular resorption in the inner portion of the stent, the reendothelialization may be disturbed when a restenosis preventing agent is present on the inner surface of the stent, so that the first coating layer is allowed to be coated only on the outer surface of the stent.

In addition, the second coating layer coated on the uppermost layer of the stent is formed on the inner and outer surfaces of the stent, that is, on the upper layer of the first coating layer coated on the outer surface of the stent and on the nano-coupling layer on the inner surface of the stent, and includes an antithrombotic agent and an anti-inflammatory agent. At this time, as the antithrombotic agent, it is possible to use one or more selected from the group consisting of aspirin, propolis, heparin, prasugrel, ticagrelor, and clopidogrel, and as the inflammation inhibitor, it is possible to use one or more selected from the group consisting of vinpocetine, N-acetyl-seryl-aspartyl-lysyl-proline (Ac-SDKP), propolis, ibuprofen, diclofenac, and naproxen. A preferably content of the drug is 5 to 500 μg. According to a specific exemplary embodiment of the present invention, as the anti-inflammatory agent included in the second coating layer, the drugs described above may be used together with a metal salt used as the anti-inflammatory agent in the first coating layer. In this case, preferable contents of the drugs are each 5 to 500 μg.

The present invention also provides a method for manufacturing a drug-eluting stent as described above. Specifically, the method for manufacturing a drug-eluting stent according to the present invention includes:

coating a nano-coupling layer including a bioactive material into which a catechol group-containing adhesive derivative is introduced on the inner and outer surfaces of a metal or polymer stent via a dipping coating method;

coating a first coating layer including a restenosis preventing agent and an anti-inflammatory agent, which are mixed with a biocompatible polymer, only on the outer surface of the stent coated with the nano-coupling layer, and

coating a second coating layer including an antithrombotic agent and an anti-inflammatory agent on the first coating layer and on the nano-coupling layer on the inner surface of the stent.

A drug-elution adjusting type stent according to the present invention may temporally and spatially control the drug release by separately coating each different drug and bioactive material on the inner and outer surfaces of the stent and adjusting the biodegradation and the release time, thereby significantly lowering side effects in the initial stage, the middle stage, and the late stage after a stent treatment. Specifically, the drug-elution adjusting stent according to the present invention may prevent thrombosis and inhibit inflammation in the initial stage after the stent treatment, and effectively prevent restenosis and inflammation in the middle stage, and induce reendothelialization and prevent thrombosis in the late stage. Further, the stability of the coating on the surface of the stent is enhanced, and cracks may be prevented from being formed by imparting flexibility to the surface.

### BRIEF DESCRIPTION OF THE DRAWINGS

FIG. 1 illustrates the drug-release rates of an antithrombotic agent and an anti-inflammatory agent from a second coating layer in the stent according to the present invention. In the drawing, ASP and VIP indicate aspirin and vinpocetine, respectively.

FIG. 2 illustrates the drug-release rates of a restenosis preventing agent from a first coating layer in the stent according to the present invention. In the drawing, the black color, the red color, and the blue color indicate the release profile of PLGA (50:50)+sirolimus, PLGA (65:35)+sirolimus, and PLGA (75:25)+sirolimus, respectively.

### MODES FOR CARRYING OUT THE PREFERRED EMBODIMENTS

Hereinafter, the contents of the present invention will be described in more detail through the Examples. The Examples are only for describing the present invention, and it will be obvious to those skilled in the art that the scope of the present invention is not interpreted to be limited by these Examples.

### Example 1

A nano-coupling layer was formed by coating a hyaluronic acid-dopamine (HA-DA) solution on the inner and outer surfaces of a cobalt-chromium stent by a dipping method. The HA-DA solution was prepared by dissolving a hyaluronic acid having a molecular weight 13 kDa at a concentration of 10 w/w % in 0.5 M of a 2-(N-morpholino)ethane sulfonic acid (MES) buffer (pH 5.5), adding dopamine at a ratio of 1 to 50 w/w % to hyaluronic acid to synthesize a product through a carbodiamide reaction, and dissolving the product in a tris buffer (pH 8.5) to produce a solution at a concentration of 0.1 to 10 mg/mL. Next, a first coating solution was prepared by dissolving a poly(lactic acid-co-glycolic acid) (PLGA, 50:50) having a molecular weight of 40 kDa at a concentration of 0.3 w/w % in a methylene chloride solvent, and then mixing 20 w/w % of sirolimus (a restenosis preventing agent) and 5 w/w % of magnesium hydroxide (an anti-inflammatory agent) of the PLGA polymer. The first coating solution was coated only on the outer surface of the stent by using an ultrasonic nano-spray coating system. At this time, the first coating solution was selectively coated only on the outer portion of the stent by embedding the inner portion of the stent with a polymer mold to prevent the inner portion of the stent from being coated with the first coating solution.

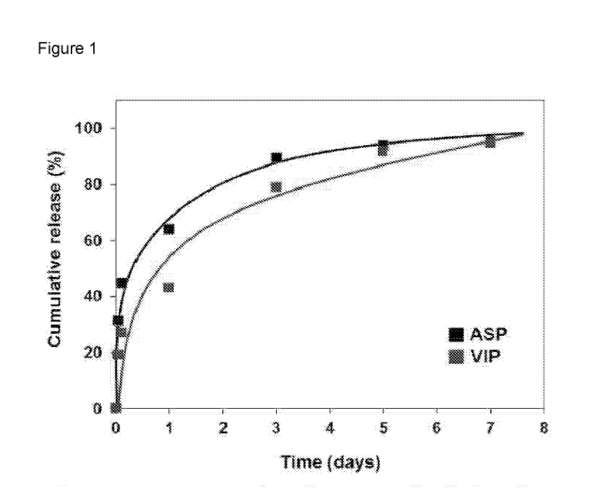

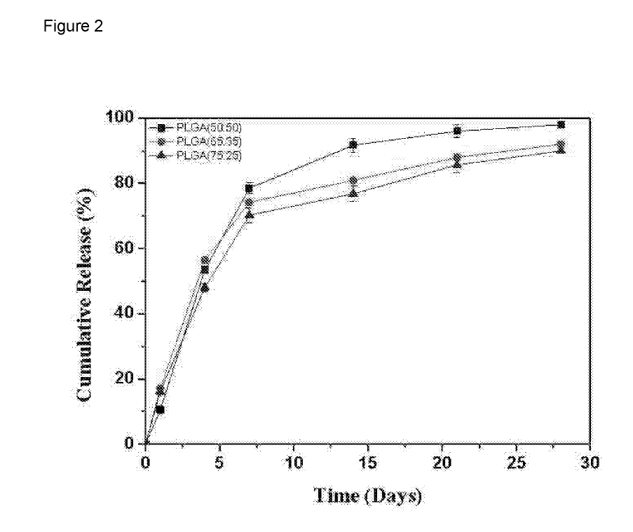

A second coating layer was prepared by dissolving a PLGA (50:50) having a molecular weight of 40 kDa at a concentration of 0.3 w/w % in a methylene chloride solvent and mixing 20 w/w % of aspirin (an initial antithrombotic agent) and vinpocetine (an anti-inflammatory agent) of PLGA with the polymer solution at a ratio of 1:1, and was coated on the upper surface of the first coating layer and on the nano-coupling layer in the stent. In order to measure the drug release, the stent coated as described above was immersed in a PBS solution at 37° C., the solution was stirred at 100 rpm, and then each solution into which the drug was released at days 1, 3, 5, 7, 14, 21, and 28 was collected to analyze the solution through a high-performance chromatography (HPLC). Through FIGS. 1 and 2 illustrating the drug-release profile over time, it was confirmed that the initial antithrombotic agent and the anti-inflammatory agent coated on the top layer had been released within 1 week, and the restenosis preventing agent had been completely released within 3 months.

### Example 2

A nano-coupling layer was formed by coating a vita-hyaluronic acid-dopamine (vita-HA-DA) on the inner and outer surfaces of a stainless steel stent by a dipping method. The vita-HA-DA solution was prepared by dissolving vitamin C-hyaluronic acid at a concentration of 10 w/w % in 0.5 M of an MES buffer (pH 5.5). Next, a first coating solution was prepared by dissolving a polymer of a poly(DL-lactide) (PDLLA) having a molecular weight of 100 kDa at a concentration of 0.3 w/w % in a chloroform solvent, and then mixing 20 w/w % of a restenosis preventing agent alpha-lipoic acid and 5 w/w % of an anti-inflammatory agent magnesium hydroxide. The first coating solution prepared as described above was coated only on the outer surface of the stent by using an ultrasonic nano-spray coating system, and at this time, the first coating solution was selectively coated only on the outer portion of the stent by embedding the inner portion of the stent with a polymer mold to prevent the inner portion of the stent from being coated with the first coating solution. A second coating solution was prepared by dissolving a biodegradable polymer PLGA (50:50) having a molecular weight of 40 kDa at a concentration of 0.3 w/w % in a chloroform solvent and mixing 20 w/w % of aspirin and 5 w/w % of vinpocetine with the polymer solution, and was coated on the upper surface of the first coating layer and on the nano-coupling layer in the stent. By the same method as in Example 1, it was confirmed that the drug had been released, and that the initial antithrombotic agent and the anti-inflammatory agent coated on the top layer had been released within 1 week, and the restenosis preventing agent had been completely released within 3 months.

### Example 3

A nano-coupling layer was formed by coating a chondroitin sulfate-dopamine (CS-DA) solution on the inner and outer surfaces of a nitinol stent by a dipping method. The CS-DA solution was prepared by dissolving a chondroitin sulfate having a molecular weight of 30 kDa at a concentration of 10 w/w % in 0.5 M of an MES buffer (pH 5.5), adding dopamine at a ratio of 1 to 50 w/w % to chondroitin sulfate to synthesize a product through a carbodiamide reaction, and dissolving the product in a tris buffer (pH 8.5) to produce a solution at a concentration of 0.1 to 10 mg/mL. Next, a first coating solution was prepared by dissolving a polymer of a poly(lactide-co-glycolic acid) (PLGA, 65:35) having a molecular weight of 80 kDa at a concentration of 0.3 w/w % in a chloroform solvent, and then mixing 20 w/w % of a restenosis preventing agent abciximab and 15 w/w % of an anti-inflammatory agent magnesium hydroxide. The first coating solution prepared as described above was coated only on the outer surface of the stent by using an ultrasonic nano-spray coating system, and at this time, the first coating solution was selectively coated only on the outer portion of the stent by embedding the inner portion of the stent with a polymer mold to prevent the inner portion of the stent from being coated with the first coating solution. A second coating solution was prepared by dissolving a PLGA (65:35) having a molecular weight of 40 kDa at a concentration of 0.3 w/w % in a methylene chloride solvent and mixing 100 units/mL of an antithrombotic agent heparin and 50 pg/mL of an anti-inflammatory agent propolis with the polymer solution, and was coated on the upper surface of the first coating layer and on the nano-coupling layer in the stent. By the same method as in Example 1, it was confirmed that the drug had been released, and that the initial antithrombotic agent and the anti-inflammatory agent coated on the top layer had been released within 1 week, and the restenosis preventing agent had been completely released within 3 months.

### Example 4

A nano-coupling layer was formed by coating a heparin-dopamine (HEP-DA) solution on the inner and outer surfaces of a magnesium metal stent by a dipping method. The HEP-DA solution was prepared by dissolving a heparin having a molecular weight of 15 kDa at a concentration of 10 w/w % in 0.5 M of an MES buffer (pH 5.5), adding dopamine at a ratio of 1 to 50 w/w % to heparin to synthesize a product through a carbodiamide reaction, and dissolving the product in a tris buffer (pH 8.5) to produce a solution at a concentration of 0.1 to 10 mg/mL. Next, a first coating solution was prepared by dissolving a poly(lactide-co-c-caprolactone) (PLCL, 85:15) having a molecular weight of 75 kDa at a concentration of 0.3 w/w % in a chloroform solvent, and then mixing 20 w/w % of a restenosis preventing agent paclitaxel and 15 w/w % of an anti-inflammatory agent magnesium hydroxide with respect to the weight of the polymer. The first coating solution prepared as described above was coated only on the outer surface of the stent by using an ultrasonic nano-spray coating system, and at this time, the first coating solution was selectively coated only on the outer portion of the stent by embedding the inner portion of the stent with a polymer mold to prevent the inner portion of the stent from being coated with the first coating solution. A second coating solution was prepared by dissolving a PLGA (50:50) polymer having a molecular weight of 40 kDa at a concentration of 0.3 w/w % in a chloroform solvent and mixing 30 μg/mL of an initial antithrombotic agent prasugrel and 30 μg/mL of an anti-inflammatory agent ibuprofen with the polymer solution, and was coated on the upper surface of the first coating layer and on the nano-coupling layer in the stent. By the same method as in Example 1, it was confirmed that the drug had been released, and that the initial antithrombotic agent and the anti-inflammatory agent coated on the top layer had been released within 1 week, and the restenosis preventing agent had been completely released within 3 months.

### Example 5

A nano-coupling layer was formed by coating a heparan sulfate-dopamine (HS-DA) solution on the inner and outer surfaces of a platinum-iridium stent by a dipping method. The HS-DA solution was prepared by dissolving a heparan sulfate having a molecular weight of 135 kDa at a concentration of 10 w/w % in 0.5 M of an MES buffer (pH 5.5), adding dopamine at a ratio of 1 to 50 w/w % to heparan sulfate to synthesize a product through a carbodiamide reaction, and dissolving the product in a tris buffer (pH 8.5) to produce a solution at a concentration of 0.1 to 10 mg/mL. Next, a first coating solution was prepared by dissolving a poly(lactide-co-ε-caprolactone) (PLCL, 70:30) having a molecular weight of 200 kDa at a concentration of 0.3 w/w % in a methylene chloride solvent, and then mixing 20 w/w % of a restenosis preventing agent sirolimus derivative and 15 w/w % of an anti-inflammatory agent magnesium fluoride with respect to the weight of the polymer. The first coating solution prepared as described above was coated only on the outer surface of the stent by using an ultrasonic nano-spray coating system, and at this time, the first coating solution was selectively coated only on the outer portion of the stent by embedding the inner portion of the stent with a polymer mold to prevent the inner portion of the stent from being coated with the first coating solution. A second coating solution was prepared by dissolving a PLGA (50:50) having a molecular weight of 40 kDa at a concentration of 0.3 w/w % in a methylene chloride solvent and mixing an initial antithrombotic agent clopidogrel and an anti-inflammatory agent diclofenac at a ratio of 1:1 at a concentration of 20 w/w % with respect to the weight of the polymer, and was coated on the upper surface of the first coating layer and on the nano-coupling layer in the stent. By the same method as in Example 1, it was confirmed that the drug had been released, and that the initial antithrombotic agent and the anti-inflammatory agent coated on the top layer had been released within 1 week, and the restenosis preventing agent had been completely released within 3 months.

### Example 6

A nano-coupling layer was formed by coating a dextran-dopamine (DEX-DA) solution on the inner and outer surfaces of a tantalum stent by a dipping method. The DEX-DA solution was prepared by dissolving a dextran having a molecular weight of 12 kDa at a concentration of 10 w/w % in 0.5 M of an MES buffer (pH 5.5), adding dopamine at a ratio of 1 to 50 w/w % to dextran to synthesize a product through a carbodiamide reaction, and dissolving the product in a tris buffer (pH 8.5) to produce a solution at a concentration of 0.1 to 10 mg/mL. Next, a first coating solution was prepared by dissolving a poly(L-lactide) (PLLA) having a molecular weight of 300 kDa at a concentration of 0.3 w/w % in a chloroform solvent, and then mixing 20 w/w % of a restenosis preventing agent tacrolimus and 15 w/w % of an anti-inflammatory agent magnesium oxide with respect to the weight of the polymer. The first coating solution prepared as described above was coated only on the outer surface of the stent by using an ultrasonic nano-spray coating system, and at this time, the first coating solution was selectively coated only on the outer portion of the stent by embedding the inner portion of the stent with a polymer mold to prevent the inner portion of the stent from being coated with the first coating solution. A second coating solution was prepared by dissolving a PLGA (50:50) having a molecular weight of 40 kDa at a concentration of 0.3 w/w % in a chloroform solvent and mixing an initial antithrombotic agent ticagrelor and an anti-inflammatory agent naproxen at a ratio of 3:2 at a concentration of 20 w/w % with respect to the weight of the polymer, and was coated on the upper surface of the first coating layer and on the nano-coupling layer in the stent. By the same method as in Example 1, it was confirmed that the drug had been released, and that the initial antithrombotic agent and the anti-inflammatory agent coated on the top layer had been released within 1 week, and the restenosis preventing agent had been completely released within 3 months.

### Example 7

A nano-coupling layer was formed by coating a dextran sulfate-dopamine (DEXS-DA) solution on the inner and outer surfaces of a biodegradable polymer PLLA stent by a dipping method. The DEXS-DA solution was prepared by dissolving a dextran sulfate having a molecular weight of 15 kDa at a concentration of 10 w/w % in 0.5 M of an MES buffer (pH 5.5), adding dopamine at a ratio of 1 to 50 w/w % to dextran sulfate to synthesize a product through a carbodiamide reaction, and dissolving the product in a tris buffer (pH 8.5) to produce a solution at a concentration of 0.1 to 10 mg/mL. Next, a first coating solution was prepared by dissolving a poly(lactide-co-glycolic acid) (PLGA, 50:50) having a molecular weight of 180 kDa at a concentration of 0.3 w/w % in a methylene chloride solvent, and then mixing 20 w/w % of a restenosis preventing agent mycophenolic acid and 15 w/w % of an anti-inflammatory agent magnesium chloride. The first coating solution prepared as described above was coated only on the outer surface of the stent by using an ultrasonic nano-spray coating system, and at this time, the first coating solution was selectively coated only on the outer portion of the stent by embedding the inner portion of the stent with a polymer mold to prevent the inner portion of the stent from being coated with the first coating solution. A second coating solution was prepared by dissolving a PLGA (50:50) having a molecular weight of 40 kDa at a concentration of 0.3 w/w % in a methylene chloride solvent and mixing 30 pg/mL of an antithrombotic agent propolis and 15 w/w % of an anti-inflammatory agent magnesium hydroxide with the polymer solution, and was coated on the upper surface of the first coating layer and on the nano-coupling layer in the stent. By the same method as in Example 1, it was confirmed that the drug had been released, and that the initial antithrombotic agent and the anti-inflammatory agent coated on the top layer had been released within 1 week, and the restenosis preventing agent had been completely released within 3 months.

### Example 8

A nano-coupling layer was formed by coating a dermatan sulfate-dopamine (DS-DA) solution on the inner and outer surfaces of a biodegradable polymer PLGA (50:50) stent by a dipping method. The DS-DA solution was prepared by dissolving a dermatan sulfate having a molecular weight of 12 kDa at a concentration of 10 w/w % in 0.5 M of an MES buffer (pH 5.5), adding dopamine at a ratio of 1 to 50 w/w % to dermatan sulfate to synthesize a product through a carbodiamide reaction, and dissolving the product in a tris buffer (pH 8.5) to produce a solution at a concentration of 0.1 to 10 mg/m L. Next, a first coating solution was prepared by dissolving a poly(DL-lactide) (PDLLA) having a molecular weight of 200 kDa at a concentration of 0.3 w/w % in a chloroform solvent, and then mixing 20 w/w % of a restenosis preventing agent estradiol and 15 w/w % of an anti-inflammatory agent magnesium hydroxide with respect to the weight of the polymer. The first coating solution prepared as described above was coated only on the outer surface of the stent by using an ultrasonic nano-spray coating system, and at this time, the first coating solution was selectively coated only on the outer portion of the stent by embedding the inner portion of the stent with a polymer mold to prevent the inner portion of the stent from being coated with the first coating solution. A second coating solution was prepared by dissolving a PLGA (50:50) having a molecular weight of 40 kDa at a concentration of 0.3 w/w % in a chloroform solvent and mixing 30 pg/mL of an initial antithrombotic agent aspirin and 50 μg/mL of an anti-inflammatory agent Ac-SDKP with the polymer solution, and was coated on the upper surface of the first coating layer and on the nano-coupling layer in the stent. By the same method as in Example 1, it was confirmed that the drug had been released, and that the initial antithrombotic agent and the anti-inflammatory agent coated on the top layer had been released within 1 week, and the restenosis preventing agent had been completely released within 3 months.

### Example 9

A nano-coupling layer was formed by coating a keratan sulfate-dopamine (KS-DA) solution on the inner and outer surfaces of a biodegradable polymer PLCL by a dipping method. The KS-DA solution was prepared by dissolving a keratan sulfate having a molecular weight of 5 kDa at a concentration of 10 w/w % in 0.5 M of an MES buffer (pH 5.5), adding dopamine at a ratio of 1 to 50 w/w % to keratan sulfate to synthesize a product through a carbodiamide reaction, and dissolving the product in a tris buffer (pH 8.5) to produce a solution at a concentration of 0.1 to 10 mg/mL. Next, a first coating solution was prepared by dissolving a poly(lactide-co-glycolic acid) (PLGA, 75:25) having a molecular weight of 120 kDa at a concentration of 0.3 w/w % in a methylene chloride solvent, and then mixing 1 mg/mL of a restenosis preventing agent nitrogen oxide and 15 w/w % of an anti-inflammatory agent magnesium oxide. The first coating solution prepared as described above was coated only on the outer surface of the stent by using an ultrasonic nano-spray coating system, and at this time, the first coating solution was selectively coated only on the outer portion of the stent by embedding the inner portion of the stent with a polymer mold to prevent the inner portion of the stent from being coated with the first coating solution. A second coating solution was prepared by dissolving a PLGA (75:25) having a molecular weight of 40 kDa at a concentration of 0.3 w/w % in a methylene chloride solvent and mixing 100 units/mL of an antithrombotic agent heparin and 30 μg/mL of an anti-inflammatory agent propolis with the polymer solution, and was coated on the upper surface of the first coating layer and on the nano-coupling layer in the stent. By the same method as in Example 1, it was confirmed that the drug had been released, and that the initial antithrombotic agent and the anti-inflammatory agent coated on the top layer had been released within 1 week, and the restenosis preventing agent had been completely released within 3 months.

### Comparative Example 1

In a cobalt-chromium stent, a restenosis preventing agent sirolimus was mixed with a PLGA (50:50) having a molecular weight of 40 kDA, and the resulting mixture was coated on the surface of the stent by using a nano-spray coating system.

### Comparative Example 2

A nano-coupling layer was formed by coating hyaluronic acid -dopamine (HA-DA) on the cobalt-chromium stent by a dipping method. A restenosis preventing agent sirolimus was mixed with a PLGA (50:50) having a molecular weight of 40 kDA, and the resulting mixture was coated on the surface of the stent by using a nano-spray coating system.

### Comparative Example 3

A nano-coupling layer was formed by coating hyaluronic acid -dopamine (HA-DA) on the cobalt-chromium stent by a dipping method. A restenosis preventing agent sirolimus was mixed with a PLGA (50:50) having a molecular weight of 40 kDA, and the resulting mixture was coated on the surface of the stent by using a nano-spray coating system.

The configurations of the drug-eluting stents in the following Examples and Comparative Examples are summarized in the following Table, and these characteristics of the stents were compared with each other, and the comparisons are also shown in the following Table.

| | | | First coating layer | | | Second coating layer | |
|---|---|---|---|---|---|---|---|
| Example/ | | Nano- | | Restenosis | anti- | | anti- |
| Comparative | Stent | coupling | Biocompatible | prevention | inflammatory | antithrombotic | inflammatory |
| Example | material | layer | polymer | agent | agent | agent | agent |
| Example 1 | Cobalt- | HA-DA | PLGA | Sirolimus | Magnesium | Aspirin | Vinpocetine |
| | Chromium | | | | hydroxide | | |
| Example 2 | Stainless | Vita-HA- | PDLLA | Alpha-lipoic | Magnesium | Aspirin | Vinpocetine |
| | steel | DA | | acid | oxide | | |
| Example 3 | Nitinol | CS-DA | PLGA | Reopro | Magnesium | Heparin | Propolis |
| | | | | | hydroxide | | |
| Example 4 | Magnesium | HEP-DA | PLCL | Paclitaxel | Magnesium | Prasugrel | Ibuprofen |
| | | | | | oxide | | |
| Example 5 | Platinum- | HS-DA | PLCL | Sirolimus | Magnesium | Clopidogrel | Diclofenac |
| | Iridium | | | derivative | fluoride | | |
| Example 6 | Tantalum | DEX-DA | PLLA | Tacrolimus | Magnesium | Ticagrelor | Naproxen |
| | | | | | oxide | | |
| Example 7 | PLLA | DEXS- | PLGA | Mycophenolic | Magnesium | Propolis | Magnesium |
| | | DA | | acid | chloride | | hydroxide |
| Example 8 | PLGA | DS-DA | PDLLA | Estradiol | Magnesium | Aspirin | Ac-SDKP |
| | | | | | hydroxide | | |
| Example 9 | PLCL | KS-DA | PLGA | Nitrogen | Magnesium | Heparin | Propolis |
| | | | | oxide | oxide | | |
| Comparative | Cobalt- | — | PLGA | Sirolimus | — | — | — |
| Example 1 | Chromium | | | | | | |
| Comparative | Cobalt- | HA-DA | PLGA | Sirolimus | — | — | — |
| Example 2 | Chromium | | | | | | |
| Comparative | Cobalt- | HA-DA | PLGA | Sirolimus | Magnesium | — | — |
| Example 3 | Chromium | | | | hydroxide | | |

| | Initial | Inflammatory | | | |
|---|---|---|---|---|---|
| Classification | thrombosis | response | Restenosis | Reendothelialization | Late thrombosis |
| Example 1 | X | X | X | ◯ | X |
| Example 2 | X | X | X | ◯ | X |
| Example 3 | X | X | X | ◯ | X |
| Example 4 | X | X | X | ◯ | X |
| Example 5 | X | X | X | ◯ | X |
| Example 6 | X | X | X | ◯ | X |
| Example 7 | X | X | X | ◯ | X |
| Example 8 | X | X | X | ◯ | X |
| Example 9 | X | X | X | ◯ | X |
| Comparative | ◯ | ◯ | X | X | Δ |
| Example 1 | | | | | |
| Comparative | ◯ | ◯ | X | ◯ | Δ |
| Example 2 | | | | | |
| Comparative | ◯ | X | X | ◯ | Δ |
| Example 3 | | | | | |

(◯: Proceeding at 70% or more, Δ: Proceeding at 50% or more, X: Proceeding at 30% or more)

What is claimed is:

1. A drug-eluting stent comprising:
a stent;
a nano-coupling layer formed on the inner and outer surfaces of the stent and including a bioactive material into which a catechol group-containing adhesive derivative is introduced;
a first coating layer formed on the nano-coupling layer on the outer surface of the stent and comprising a restenosis preventing agent and an anti-inflammatory agent, which are mixed with a biocompatible and biodegradable polymer; and
a second coating layer formed on the first coating layer on the outer surface of the stent and on the nano-coupling layer on the inner surface of the stent, and comprising an antithrombotic agent and an anti-inflammatory agent, which are mixed with a biocompatible and biodegradable polymer.

2. The drug-eluting stent of claim 1, wherein the stent is a metal material selected from the group consisting of stainless steel, cobalt-chromium, platinum-chromium, tantalum, titanium, nitinol, platinum-iridium, iron, gold, platinum, silver, magnesium, and alloys thereof, or a polymer material selected from the group consisting of poly(glycolic acid) (PGA), poly(L-lactic acid) (PLLA), poly(D-lactic acid) (PDLA), poly(D,L-lactic acid) (PDLLA), poly(c-caprolactone) (PCL), poly(lactic acid-co-glycolic acid) (PLGA), poly(L-lactic acid-co-c-caprolactone) (PLCL), tyrosine polycarbonate, salicylic acid-containing polymers, polyethylene glycol, polyamino acid, polyanhydride, polyorthoester, polydioxanone, polyphosphazene, cellulose acetate butyrate, cellulose triacetate, and copolymers thereof.

3. The drug-eluting stent of claim 1, wherein the bioactive material of the nano-coupling layer is selected from the group consisting of hyaluronic acid, acetylated hyaluronic acid, chondroitin sulfate, heparin, heparan sulfate, dextran, dextran sulfate, dermatan sulfate, and keratan sulfate and, vita-hyaluronic acid, vita-acetylated hyaluronic acid, vita-chondroitin sulfate, vita-heparin, vita-heparan sulfate, vita-dextran, vita-dextran sulfate, vita-dermatan sulfate, and vita-keratan sulfate mean that any one of vitamin A, a vitamin B complex, vitamin C, vitamin D, vitamin E, vitamin F, vitamin K, vitamin U, vitamin L, and vitamin P each combines with hyaluronic acid, acetylated hyaluronic acid, chondroitin sulfate, heparin, heparan sulfate, dextran, dextran sulfate, dermatan sulfate, or keratan sulfate.

4. The drug-eluting stent of claim 1, wherein the catechol group-containing adhesive derivative is selected from the group consisting of dopamine, norepinephrine, epinephrine, 3,4-dihydroxybenzylamine, 3,4-dihydroxycinnamic acid, 3,4-dihydroxyphenyl acetic acid, 3,4-dihydroxymandelic acid, 3,4-dihydroxyphenyl lactic acid, 3,4-dihydroxyphenylalanine, 2-(3,4-dihydroxyphenyl)ethanol, 3,4-dihydroxyphenylethyleneglycol, 3,4-dihydroxyphenylacetaldehyde, 3,4-dihydroxyphenylglycol aldehyde, and isoproterenol.

5. The drug-eluting stent of claim 1, wherein the biocompatible polymer of the first coating layer is a polymer having a molecular weight in a range of 5,000 to 500,000, selected from the group consisting of polyvinyl alcohol, polyethylene glycol, polylactide, polyglycolide, polylactide copolymers, polycaprolactone copolymers, polyethylene oxide, polydioxanone, and polyvinylpyrrolidone.

6. The drug-eluting stent of claim 1, wherein the restenosis preventing agent of the first coating layer is selected from the group consisting of alpha-lipoic acid, Abciximab, sirolimus, sirolimus derivatives, paclitaxel, dexamethasone, tacrolimus, mycophenolic acid, estradiol, taxol, colchicine, lovastatin, trapidil, hirudin, ticlopidine, and nitrogen oxides.

7. The drug-eluting stent of claim 1, wherein the anti-inflammatory agent of the first coating layer and the second coating layer is selected from the group consisting of magnesium, magnesium oxide, magnesium fluoride, magnesium chloride, magnesium hydroxide, lithium hydroxide, beryllium hydroxide, sodium hydroxide, potassium hydroxide, rubidium hydroxide, strontium hydroxide, barium hydroxide, cesium hydroxide, francium hydroxide, and radium hydroxide.

8. The drug-eluting stent of claim 1, wherein the anti-inflammatory agent of the second coating layer is one or more selected from the group consisting of vinpocetine, Ac-SDKP, propolis, ibuprofen, diclofenac, and naproxen.

9. The drug-eluting stent of claim 1, wherein the antithrombotic agent of the second coating layer is one or more selected from the group consisting of aspirin, propolis, heparin, prasugrel, ticagrelor, and clopidogrel.
